(19) United States
(12) Patent Application Publication
(10) Pub. No.: US 2009/0312667 A1
(43) Pub. Date: Dec. 17, 2009

(54) Birth Prediction Reporting System for Live Stock Animals

(75) Inventors: Shigeo Utsunomiya, Oita (JP); Tetsu Ikeda, Oita (JP); Shuichi Takeishi, Oita (JP)

Correspondence Address: RADER FISHMAN & GRAUER PLLC, LION BUILDING, 1233 20TH STREET N.W., SUITE 501, WASHINGTON, DC, 20036, US

(73) Assignee: Remote Inc., Oita (JP)

(21) Appl. No.: 12/226,742

(22) PCT Filed: Apr. 27, 2007
(86) PCT No.: PCT/JP2007/059225

§ 371 Date: Mar. 6, 2009

(30) Foreign Application Priority Data
Apr. 28, 2006 (JP) 2006-125231

(51) Int. Cl. A61B 5/01

(52) U.S. Cl. 600/549

(57) ABSTRACT

A system of accurately predicting birth of large-sized livestock such as cows sufficiently before the time of making delivery preparations and attending delivery is provided. Vaginal temperatures of livestock such as cows measured by temperature sensor/transmission modules 100 inserted in the vagina of the livestock are transmitted wirelessly, received by a reception module 200, and sent to a monitor center 350 from a station 310 connected to the Internet 300 via a wireless LAN or a wired LAN. The monitoring center 350 is capable of storing the transmitted vaginal temperature data of each livestock, predicting birth timings from the stored vaginal temperature data, and reporting the birth timings by email or the like to a feeder's cellular phone 320 or a cellular phone 340 of an affiliated veterinarian.

### TECHNICAL FIELD

The present invention relates to a birth prediction reporting system capable of informing that birth of large-sized livestock such as cows is near.

### BACKGROUND ART

Knowing the delivery timing of large-sized livestock such as cows is important for a feeder in order to prevent accidents at the time of delivery by attending the delivery. For example, in the case of artificially inseminating cows, the expected delivery date is 285 days after the artificial insemination date. However, there are individual differences, and a calf is not always born on the expected delivery date. Moreover, in the case of group feeding livestock such as cows, and the like, it is difficult to attend expecting livestock to observe indications of delivery.

Therefore, a system for predicting various birth timings, which allows sufficient, conventional preparations for delivery, has been considered. A system that centrally controls at a livestock control center by attaching sensors to livestock and wirelessly transmitting measured data to a transponder is also proposed (Refer to Patent Document 1).

However, although this system utilizes measured data of at least one of respiratory rate, blood pressure, and body temperature, what state indicates possible birthing is unclear, and thus exact prediction is impossible.

In addition, prediction of birth based on finding that a cordless monitor inserted in the birth canal has come out due to rupture of the allantoic membrane before birth, which is obtained by detecting water or change in temperature, has also been proposed (Refer to Patent Documents 2 and 3). However, since sufficient preparation for the delivery cannot be made during the period from detection of membrane rupture to delivery, accurate delivery prediction at a much earlier time has been desired. Report of birth prediction is desirably a prediction indicating that membrane rupture or birth will start in 1 to 2 days.

Patent Document 1: International Publication for PCT Application No. WO01/80630

### Patent Document 2: Japanese Unexamined Patent Application Publication No. 2005-110880

### Patent Document 3: Japanese Unexamined Patent Application Publication No. 2005-261686

### DISCLOSURE OF INVENTION

### Problems to be Solved by the Invention

An objective of the present invention is to provide a system for accurately predicting birth of large-sized livestock such as cows sufficiently before the time of making delivery preparations and then attending delivery.

### Means of Solving the Problems

In order to achieve the aforementioned objective, the present invention is characterized by a birth prediction reporting system, comprising: a temperature sensor/transmission module, which is inserted in the vagina of a cow, measures vaginal temperature, and transmits wirelessly the measured vaginal temperature along with its own ID; a reception module, which receives transmission data based on the temperature sensor/transmission module and sends the received data via a communication line; and a monitor center, which receives data from the reception module via the communication line, predicts birth from a vaginal temperature, and reports it. The temperature sensor/transmission module measures with high accuracy within a specific range in the vicinity of average body temperature, and the monitor center measures a moving-average value of vaginal temperatures within a predetermined period of time for each livestock is measured based on the ID, compares the moving-average with previous moving-averages to predict birth, and reports birth prediction results via the communication line.

Moreover, the monitor center may determine detection of a value outside of a specified range as the temperature sensor/transmission module being pushed out of the vagina due to membrane rupture or birth, and report membrane rupture or birth via the communication line.

Furthermore, the temperature sensor/transmission module also measures a wide range of temperatures and transmits them wirelessly along with an identifier indicating a characteristic of temperature measurement, and the monitor center corrects the sent temperature based on the identifier indicating the characteristic of the temperature measurement.

When the livestock are cows, the temperature sensor/transmission module should measure with high accuracy within a range from 34.1° C. to 44.0° C., and the monitor center should measure a 4-hour moving-average value, compare it with both of the 4-hour moving-average value obtained 24 hours before and 4-hour moving-average value obtained 48 hours before, and report birth prediction when vaginal temperatures of both have decreased at least 0.3° C.

A temperature sensor/transmission module, which is inserted in the vagina of livestock, measures vaginal temperature and transmits wirelessly the measured vaginal temperature along with its own ID, preferably has an externality with at least two arms extending out from a main body where the arms are made of an elastic material, and when this module is inserted in the vagina, it fastens within the vagina by the arms pressing against the vaginal inner wall, and is pushed out of the vagina due to membrane rupture or birth. It is also preferable that an antenna with a conductive wire extends from between the arms to the outside of the cow body.

The temperature sensor/transmission module may include a main body and a stopper ring fixed to the main body. The stopper ring is made of an elastic material, and may include a ring portion and at least two arms extending from the ring portion, and the ring portion of the stopper ring may fit into the main body.

The antenna may include a resin ball for escape prevention at the tip thereof.

### Results of Invention

Since the birth prediction reporting system of the present invention utilizes calculated moving-average values, noise of vaginal temperature detection may be eliminated. Therefore, accurate birth prediction may be provided, and reporting the prediction with sufficiently extra time is possible. Moreover, membrane rupture and birth may also be reported.

A wide range of temperatures such as ambient temperature and water temperature may also be measured with sufficient accuracy through correction according to characteristics of each temperature sensor/transmission module.

In the case of cows, reporting with sufficiently extra time is possible by measuring a 4-hour moving-average value, comparing it with the 4-hour moving-average value obtained 24 hours before and 4-hour moving-average value obtained 48 hours before, and reporting birth prediction when vaginal temperatures of both have decreased at least 0.3° C.

The externality of a temperature sensor/transmission module inserted in a vagina has at least two arms extending out from a main body where the arms are made of an elastic material, and when is the module inserted in the vagina, it fastens within the vagina by pressing against the vaginal inner wall by the arms, and is pushed out of the vagina due to membrane rupture or birth, thereby allowing accurate measurement of vaginal temperature and detection of membrane rupture and the like.

The antenna extending from between the arms is inserted into the main body as the shape thereof changes to be close to an I shape as possible at the time of insertion so that it can get out from the body of the livestock easily, and expands within the vagina.

### BRIEF DESCRIPTION OF DRAWINGS

FIG. 1 shows an outline of the entire configuration of a system according to the present invention;

FIG. 2-1 is a diagram showing the external appearance of a temperature sensor/transmission module used in a working example;

FIG. 2-2 is a diagram showing the external appearance of a temperature sensor/transmission module used in a working example;

FIG. 3 is a block diagram showing the entire configuration of the temperature sensor/transmission module;

FIG. 4 is a block diagram of an IC, which is a substantial part of the temperature sensor/transmission module;

FIG. 5 is a diagram showing a transmission frame format from the temperature sensor/transmission module;

FIG. 6 is a block diagram showing a configuration of a reception module;

FIG. 7 is a flowchart showing emergency signal processing at a monitor center;

FIG. 8-1 is a flowchart showing data processing at the monitor center;

FIG. 8-2 is a flowchart showing subsequent processing at the monitor center;

FIG. 9 is a table summarizing a working example in which birth of milking cows was predicted;

FIG. 10 is a table for explaining temperature correction of low-precision temperature measurement;

FIG. 11 shows a temperature sensor/transmission module, which has an escape preventing arm portion separated from the main body; wherein (a) is an external view of the main body, (b) is a top view of a stopper ring, and (c) is a side view of the stopper ring; and

FIG. 12 is a diagram showing an example of the stopper ring fixed to the main body of the temperature sensor/transmission module.

### BEST MODE FOR CARRYING OUT THE INVENTION

An embodiment of the present invention is described in detail using the appended drawings.

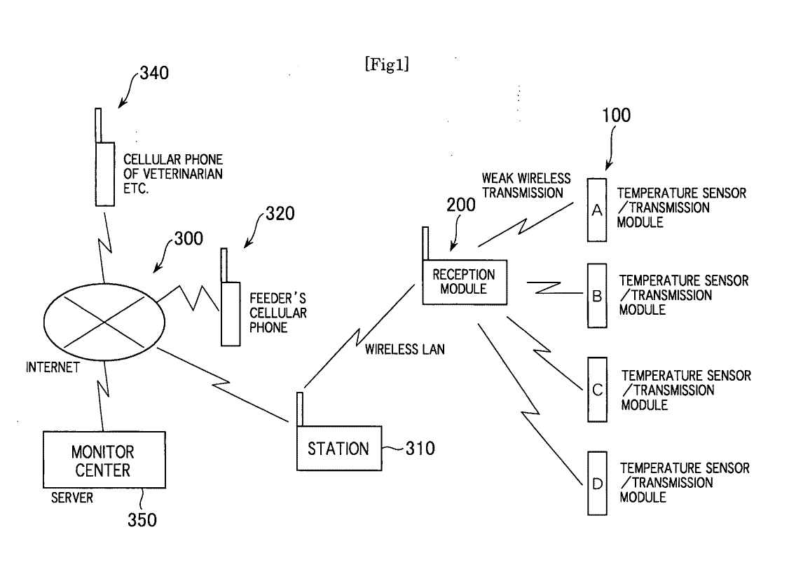

FIG. 1 shows an outline of the entire configuration of a system according to the present invention. In FIG. 1, vaginal temperatures of livestock, such as cows, measured by temperature sensor/transmission modules 100 inserted in the vaginas of the livestock are wirelessly transmitted, received by a reception module 200, and sent from a station 310, which is connected to the Internet 300, to a monitor center 350 via a wireless LAN or a wired LAN.

The IDs of the respective temperature sensor/transmission modules 100 and the same measurement results are transmitted five times from the respective temperature sensor/transmission modules 100 at random intervals during a period of approximately five minutes. When measurement results are transmitted from the respective multiple temperature sensor/transmission modules 100, they are converged and received by the reception module 200. However, if the reception module 200 receives multiple transmission signals simultaneously, it cannot receive them properly. Transmission at random intervals aims to minimize overlapped reception.

The vaginal temperatures received by the reception module 200 are each given a reception module 200 ID and sent to the monitor center 350 via the Internet. In the case where many cows are grazing in a large pastureland, use of these reception module 200 IDs allows finding of which one of multiple installed reception modules 200 have been received. This therefore allows prediction of location of cows inserted with a temperature sensor/transmission module 100 based on the location of the reception module.

The monitor center 350 is capable of storing the transmitted vaginal temperature data of each cow, predicting birth timings from the stored vaginal temperature data, and sending a warning of the birth timings by email or the like to a feeder's cellular phone 320 or a cellular phone 340 of an affiliated veterinarian. While the method of determining the birth timings will be explained in detail later, it results from the inventors of this application having examined many examples and having found optimal conditions. Note that an example of reporting a warning to a cellular phone is given; however, the notification may also be sent to a personal computer connected to the Internet. Moreover, the feeder has a personal computer (omitted from the drawing) connected to the Internet so as to inspect the data stored at the monitor center and conduct various management of the monitor center.

An exemplary system or working example of a configuration of the aforementioned respective modules constructed in order to actually predict birth timings of cows is explained in detail forthwith.

### <Temperature Sensor/Transmission Module>

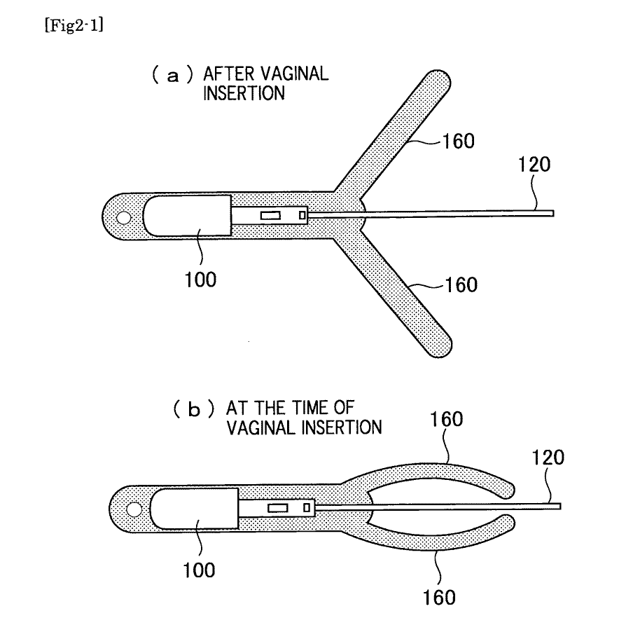

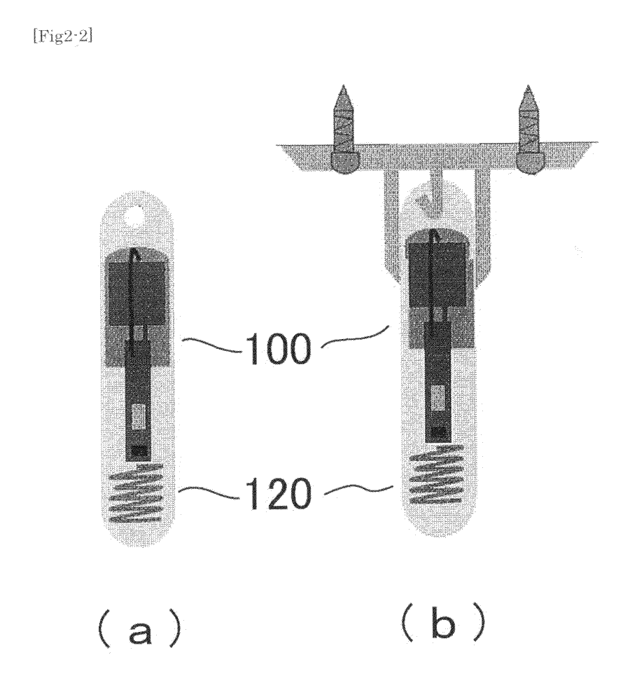

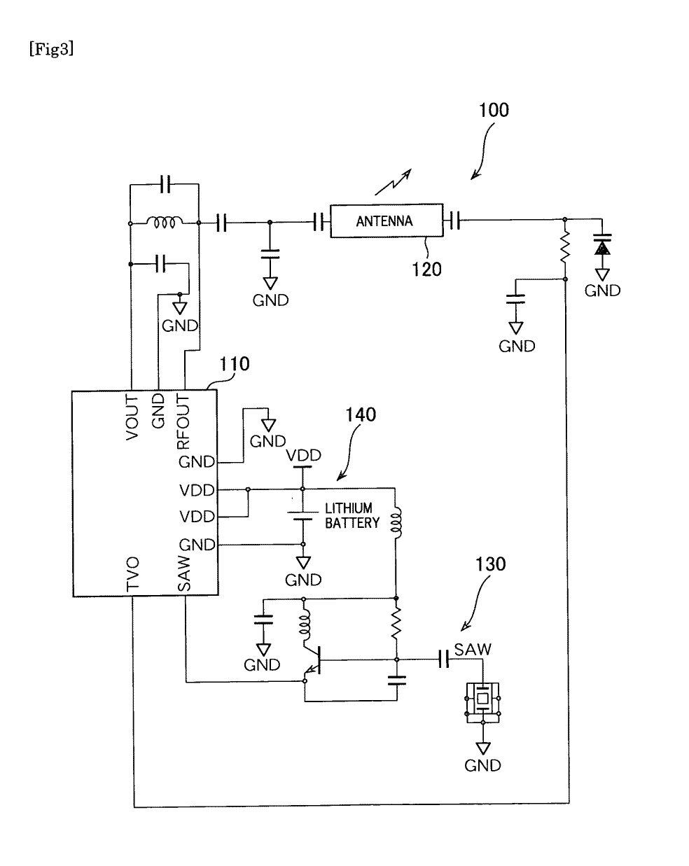

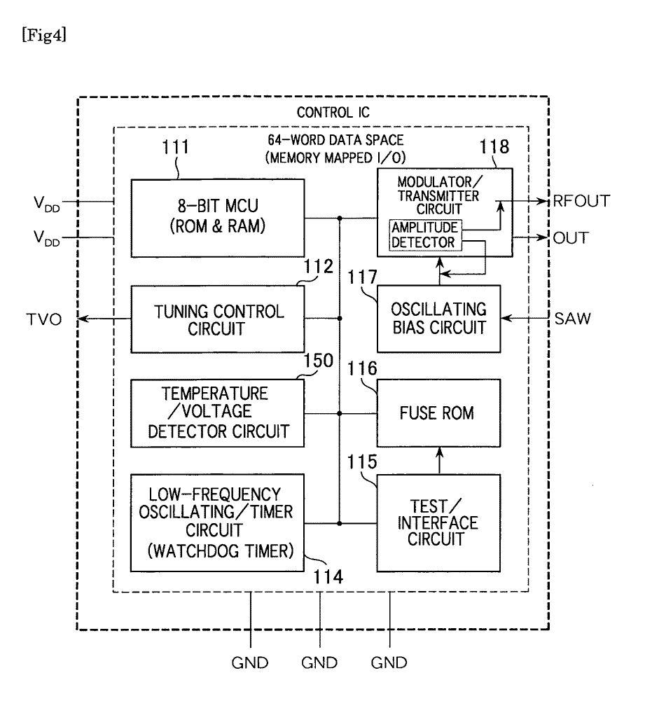

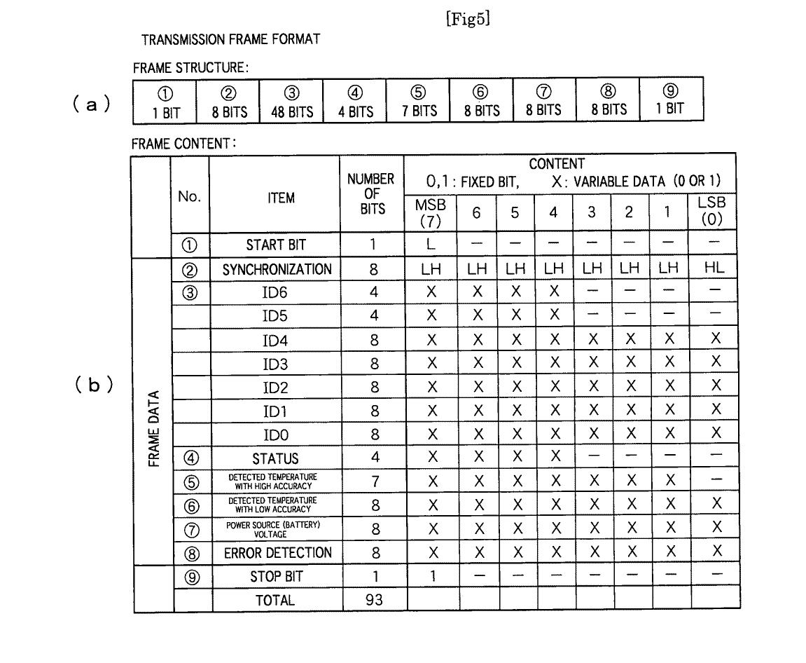

The temperature sensor/transmission module 100 is described in detail using FIGS. 2 to 5. FIG. 2 shows external appearances of the temperature sensor/transmission module 100 used in a working example of this system, and FIGS. 3 and 4 are block diagrams of the temperature sensor/transmission module 100. FIG. 5 is a diagram showing an exemplary transmission frame format output from the temperature sensor/transmission module 100.

### (External Appearance)

FIG. 2-1 is a diagram showing the external appearance of the temperature sensor/transmission module 100 used in the working example of this system. FIG. 2-1(a) shows the state after insertion into the vagina, and FIG. 2-1(b) shows the state during insertion into the vagina. As shown in FIGS. 2-1(a) and (b), the entirety is covered by an elastic material, and has a Y shape with arms on both sides. A conductive antenna 120 extends from between arms 160 to the outside of the vagina. The main body is inserted into the straight portion of the Y shape, and is water proofed by the elastic material. The arms 160 extending to either side are made by only the elastic material.

When inserting, the arms which extend on either side are straightened (see FIG. 2-1(b)) so the entirety is deformed to be an approximate I shape and inserted into the vagina and the arms 160 are extended after insertion (see FIG. 2-1(a)). As a result, due to the extending elastic arms 160 pushing against the inner walls of the vagina after insertion, the temperature sensor/transmission module 100 is prevented from easily escaping from the vagina.

While there are two arms 160 in a Y shape in the working example, there may be three or more arms 160. Polypropylene (PP), for example, is preferably used as the elastic material.

The temperature sensor/transmission module 100 with such an external appearance is not only used for birth prediction, but may be utilized for health management and the like of livestock by measuring body temperature.

Note that as described later, the temperature sensor/transmission module 100 has a configuration allowing not only precise measurement of the range of vaginal temperatures of cows but measurement of a wide range of temperatures such as ambient temperature, water temperature, and the like. While measurement of a wide range of temperatures is possible even with a Y-shaped outer shape as in FIG. 2-1(a), the external appearance of when measuring ambient temperature and the like is not a Y-shape but coiled as in FIG. 2-2(a) so as not to let the antenna 120 get outside. FIG. 2-2(b) shows the module

### (Circuit Structure of Temperature Sensor/Transmission Module)

FIG. 3 is a block diagram showing a circuit structure of the temperature sensor/transmission module 100. A control IC 110 is an IC consisting in the center of the temperature sensor/transmission module 100, and all functions of the temperature sensor/transmission module 100 such as oscillation, temperature measurement, transmission and control are performed by this IC. A lithium battery 140 as a power source, a surface acoustic wave (SAW) module 130, which sets the oscillation frequency, and the antenna 120 are mounted on this control IC 110. Note that in the working example to be described in detail, data of temperature measured using a weak signal of 315 MHz (typ.) is transmitted.

FIG. 4 is a block diagram showing an internal structure of the control IC 110.

The control IC 110 includes an 8-bit micro control unit (MCU) 111, which includes ROM and RAM and controls the entirety, a tuning control circuit 112, which feeds back a signal from the antenna to control transmission output, a temperature/voltage detector circuit 150, a low-frequency oscillating/timer circuit 114, which also functions as a watchdog timer, an interface circuit 115 for interfacing with the outside, fuse ROM 116, which is externally settable, an oscillating circuit 117, which oscillates high frequency waves and is connected to the SAW module 130, and a modulator/transmitter circuit 118, which modulates the high frequency waves according to data and transmits them.

The MCU 111 is connected to the respective units via a bus, and performs setting and control of the respective units according to a program stored in the integrated ROM.

The temperature/voltage detector circuit 150 includes a temperature sensor and a power source voltage detector. The temperature sensor precisely measures temperature in the vicinity of a cow body, and also measures a wide range of temperatures. The power source voltage detector detects voltage of the lithium battery 140, which is used as the power source, using a successive approximation type A/D converter.

IDs for respective temperature sensor/transmission modules 100 and numbers for temperature correction (described in detail later), and various motion control values are stored in the fuse ROM 116. 12 bytes may be stored in the fuse ROM 116 in the working example.

The interface circuit 115 is capable of inputting binary signals from the outside and transmitting those signals. Signals input from the outside are not transmitted in the working example described here.

The modulator/transmitter circuit 118 modulates a carrier wave oscillated by the oscillating circuit 117 according to data such as the temperature data and transmits it from the antenna 120.

Using these circuits, vaginal temperature of the cows inserted therewith is measured once every five minutes, and the data attached with the temperature sensor/transmission module 100 ID is then transmitted five times at random intervals within a period of approximately five minutes. In the working example, the carrier wave is 315 MHz (typ.), the modulation scheme is frequency-shift keying (FSK), and the communication speed is 2.5 bps.

In the working example of predicting birth timings of cows, the temperature/voltage detector circuit 150 measures cow temperatures with resolution of 0.1° C. (±0.2° C. accuracy) in the range of 34.1° C. to 44.0° C., which is the neighborhood of 38.5° C. or the basic body temperature of cows, and ambient temperature or the like (−20° C. to 60° C.) with resolution of 0.5° C. (±0.1° C. accuracy).

### (Transmission Frame Format)

FIG. 5 shows a transmission frame format transmitted from the temperature sensor/transmission module in the working example. FIG. 5(a) shows a frame structure sent in a single transmission, and FIG. 5(b) shows a breakdown of the frame.

As shown in FIG. 5(a), 93 bits comprising a frame is transmitted in a single transmission. The structure thereof is shown in FIG. 5(b). First, a 1-bit start bit and 8 bits for synchronization with the reception module 200 are assigned. Next, IDs of the temperature sensor/transmission modules 100, identification numbers for temperature correction (described later) and the like are

2 bits of a status (4 bits) are used to display a transmission counter, 1 bit of the same for over flow/under flow, and 1 bit of the same for power source abnormality.

The transmission counter (2 bits) is counted up once the same data is sent in a period of approximately 5 minutes. The transmission counter has one of values 0 to 3, which is counted up repeatedly.

The power source abnormality (1 bit) becomes 1 when the voltage of a Vₚₚ terminal of the control IC 110 is measured and falls below a preset value.

Detected temperature with high accuracy is a 7-bit binary digit X, where the binary digit X denotes {(X/10)+34}[° C.].

Detected temperature with low accuracy is an 8-bit binary digit Y, where the binary digit Y denotes {Y/0.5−40}[° C].

Power source voltage is an 8-bit binary digit Z, where the binary digit Z denotes Z×18−[mV].

Error detection is 8 bits and denotes an error detection signal of the aforementioned data. A stop bit ‘1’ is then attached at the end.

In the aforementioned manner, the temperature sensor/transmission module 100 precisely measures temperature near the body, collects that data in a frame-by-frame format, and transmits the same data five times within a period of approximately five minutes to the reception module 200.

With such a structure allowing precise measurement of body temperature, accurate prediction of birth given below has become possible.

Moreover, measurement of a wide range of temperatures is possible, and precise measurement of body temperature and measurement of common temperatures such as ambient temperature may be performed by temperature sensor/transmission modules 100 with the same structure. Therefore, ambient temperature may be measured by a temperature sensor/transmission module 100 installed not in the vagina but externally, and water temperature may be measured when it is installed under water. In this case, the Y shape as shown in FIG. 2 is not required, and attachment of the arms 160 of the Y-shape is not required. Moreover, the antenna 120 is not required to be on the outside (see FIG. 2-2).

### <Reception Module>

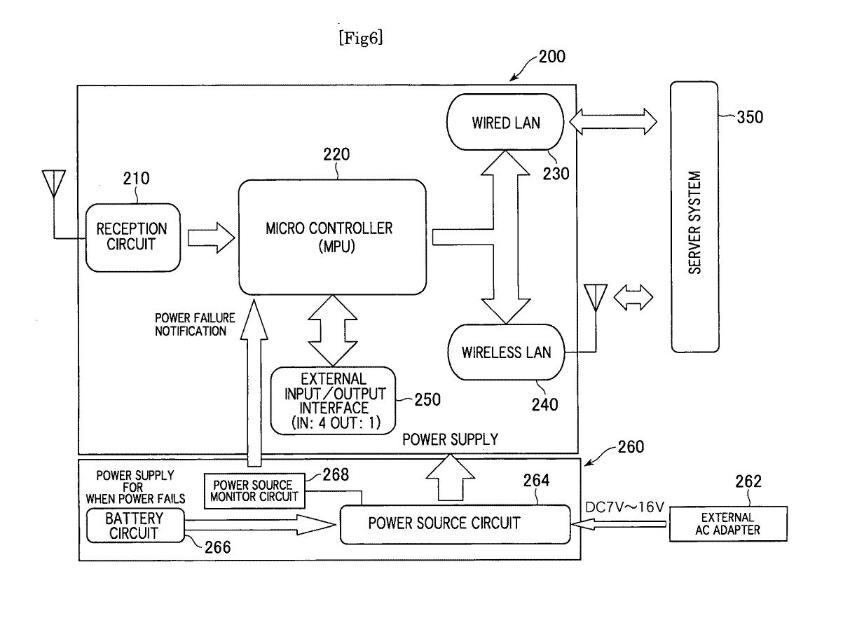

FIG. 6 is a block diagram of the reception module 200, which receives vaginal temperature data of cows from the temperature sensor/transmission module 100 and relays it to the monitor center 350 via the Internet. The reception module 200 is described in detail using FIG. 6. The reception module 200 is connected to the Internet 300 via a wired or wireless LAN by utilizing a station 310 connected to ADSL or an optical cable as a relay (see FIG. 1).

In FIG. 6, the reception module 200 includes a reception circuit 210, which receives and modulates a signal from the temperature sensor/transmission module 100, a micro controller (MPU) 220, which controls the entirety, an external input/output interface circuit 250 for inputting and outputting signals to the outside, a wired LAN port 230 and a wireless LAN port 240, which interface to the Internet, and a power source 260, which supplies electric power to each unit. The wired LAN port 230 is a port for wired LAN configured by Ethernet (registered trademark), for example. The wireless LAN port may be one of various standards such as IEEE802.11a, IEEE802.11b, or IEEE801.11g.

The reception circuit 210 receives a frame transmitted from the temperature sensor/transmission module 100, demodulates it, and sends it to the MPU 220 as digital data.

The MPU 220 performs error detection using an error detection signal for the sent data, and when there is no error, attaches a unique ID for the respective reception modules, further attaches data from the external interface 250, and sends it to the wired LAN port 230 or wireless LAN port 240.

The external input from the external interface may be a switching signal for a barn door, a signal for whether there or not there is water in a tank, or the like depending on where the reception module 200 is installed.

Sent data is transmitted through the wired LAN port 230 or wireless LAN port 240 to the internally preset IP address of the monitor center 350 via the Internet.

The power source circuit 260 supplies electric power to the respective units of the reception module 200 using a constant DC voltage from an external AC adaptor 262. It also includes a battery circuit 266 for supplying a power source during a power failure, thereby electricity may be supplied for a given length of time even if the alternator fails. Note that when electricity can no longer be supplied from the power source circuit 264, a power source monitor circuit 268 sends a power failure notification signal to the MPU 220, which is then sent to the monitor center 350 as an emergency signal separate from the signal transmitting the temperature data.

Also note that in the working example of predicting birth timings of cows, external input data is not sent from the external input/output interface 250. Moreover, external input data may be sent as an emergency signal.

### <Processing at Monitor Center>

The vaginal temperature measured by the temperature sensor/transmission module 100 inserted in the cow is sent to the monitor center 350, which then carries out processing for birth timing prediction based on the sent data and notifies prediction of birth to the cellular phone 320 of the feeder or owner of the cow via the Internet 300 (see FIG. 1).

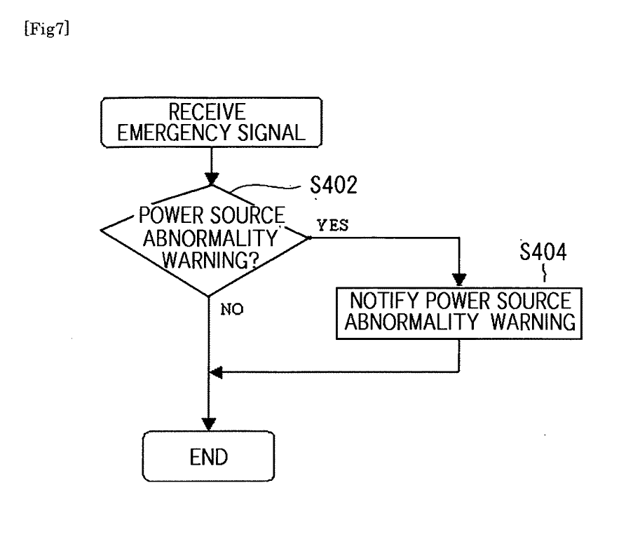

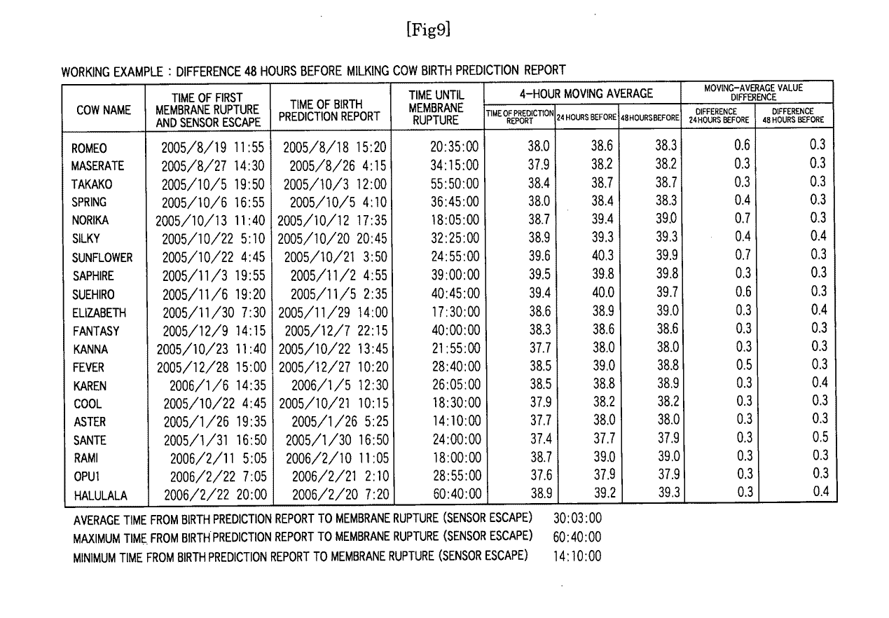

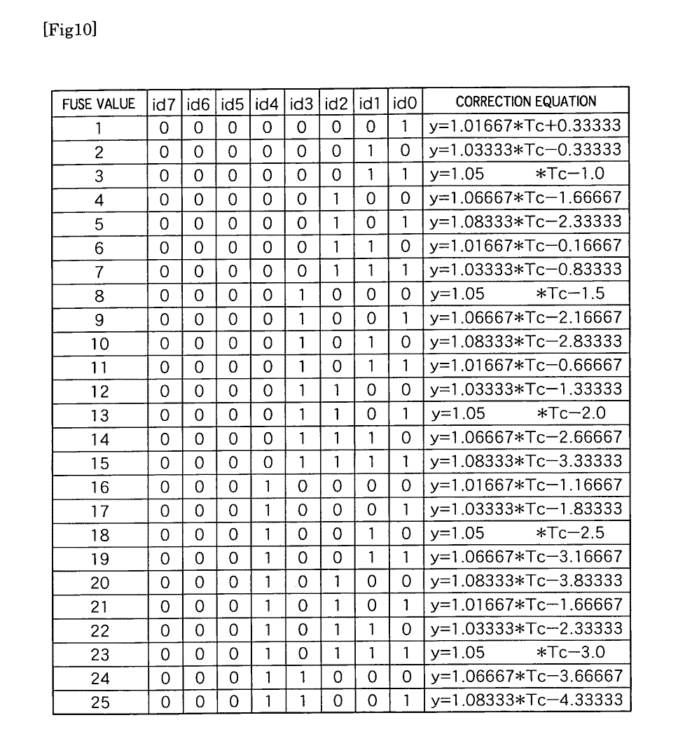

The processing carried out at the monitor center 350, which is a server on the Internet, is described in detail using FIGS. 7 to 10. FIGS. 7, 8-1 and 8-2 are flowcharts showing processing at the monitor center; FIG. 9 is a table showing results of inserting a temperature sensor/transmission module in twenty milking cows, actually measuring vaginal temperature, and predicting according to a birth predicting method described later while FIG. 10 is a table of correction equations used for low-accuracy measurement of a wide range of temperatures.

When carrying out birth prediction, as mentioned above, since in the case of cows, for example, the expected delivery date is 285 days after copulation, the temperature sensor/transmission modules 100 are inserted and held in the cow vaginas 14 days before the respective expected delivery dates. In order to determine which of the temperature sensor/transmission modules 100 has measured the vaginal temperature, the IDs of the temperature sensor/transmission modules 100 measuring vaginal temperature are pre-registered at the monitor center 350.

Ambient temperature is measured by transmitting the data of the cow vagina temperatures from the temperature sensor/transmission modules 100 to the monitor center 350.

The flowchart of FIG. 7 shows processing carried out when an emergency signal from the reception module 200 is received by the monitor center 350. When the emergency signal from the reception module 200 is received by the monitor center 350, inspect whether or not a power source abnormality of the reception module 200 has been notified (S402). For example, in the case of a power source abnormality such as power failure (YES in S402), report a power source abnormality warning to the cellular phone 320 of the feeder by email or the like (S404) and complete the signal processing.

The flowcharts of FIGS. 8-1 and 8-2 show processing to be carried out when data of temperatures measured by the temperature sensor/transmission modules 100 is received by the monitor center 350. When the data is relayed from the reception module 200 and received by the monitor center 350, first inspect whether or not the power source voltage value included in the received data is 2.0V or greater (S502). When the power source voltage value of the temperature sensor/transmission module 100 is less than 2.0V (YES in S502), display a warning of power source voltage decrease on a monitor at the monitor center 350 (S504).

As described above, the temperature sensor/transmission modules 100 of this working example detect cow vaginal temperature approximately every five minutes and transmit the same data five times during that period of approximately five minutes. In the working example, the same data is transmitted five times randomly between 280 to 310 seconds. Since the monitor center 350 will receive the same data between 280 and 310 seconds, the reference period for selecting a representative value of the vaginal temperatures is set to five minutes. While receiving data during the five minute period (NO in S506), the monitor center 350 only stores the received data in a temporary work memory area (S508).

When the beginning of a new five minute period approaches, check the sent IDs of the temperature sensor/transmission modules 100 so as to determine whether it is either a measurement of vaginal temperature or another temperature measurement, and determine whether the temperature data is within the range of 34.1° C. to 44.0° C. (S510). In the case of vaginal temperature measurement (YES in S510), read out multiple measurement results from the work memory area, and pick up and set the most frequent vaginal temperature data as a representative value out of the multiple pieces of vaginal temperature data (S512).

Next, detect membrane rupture or birth (S514). This is conducted by detecting that the temperature sensor/transmission module 100 has been pushed out from the vagina due to membrane rupture or birth. The temperature sensor/transmission module 100 being pushed out of the vagina is detected by finding whether the temperature falls below the vaginal temperature minimum value (described later) preset by the feeder (YES in S514). Notify the cellular phone 320 of the feeder or the like by email or the like of membrane rupture or birth (S516).

The feeder accesses and manages the vaginal temperature data or the like using a personal computer or the like connected to the monitor center 350 via the Internet 300. The feeder sets a vaginal temperature maximum value, vaginal temperature minimum value, report waiting period, and the like, which are references that allow the personal computer to judge whether to report. The report waiting period is reported when there is information to be reported continuously at a predetermined time. In the working example, the vaginal temperature maximum value is set to 41.0° C. while the vaginal temperature minimum value is set to 35.0° C. Moreover, these may be set from the monitor center 350.

With this system, birth is predicted by comparing moving-average data with previous data and finding the fact that the difference therebetween exceeds a predetermined value. Moving-average data is used in order to remove noise occurring in vaginal temperature detection and accurately predicting birth. Moreover, it is further useful for accurate prediction of birth to carry out comparison of the former moving-average data with each of two separate pieces of data obtained in two different previous periods.

In this working example, birth prediction is carried out using 4-hour moving-average data of cow vagina temperature, 4-hour moving-average data obtained 24 hours before, and the same obtained 48 hours while in the case of cows, birth prediction is carried out by finding the fact that it decreases at least 0.3° C. FIG. 9 shows a chart of results of birth prediction for twenty milking cows under these conditions. As can be seen from this, birth prediction is carried out for all of the twenty milking cows the earliest 14 hours before the temperature sensor/transmission modules 100 are pushed out of the body due to membrane rupture or the like. This allows sufficient preparation for birthing.

In order to carry out such prediction, the monitor center 350 calculates a 4-hour moving average (S518), and stores the representative value and the calculated 4-hour moving average for each temperature sensor/transmission module 100 ID (S520). Then determine whether or not the 4-hour moving-average data has decreased at least 0.3° C. lower than each of 4-hour moving-average data obtained 24 hours before and 4-hour moving-average data obtained 48 hours before in the case of cows (S522). When the conditions are satisfied (YES in S522), send a report of the birth prediction to the cellular phone or personal computer of the feeder or the like (S524) and conclude the processing of the received data.

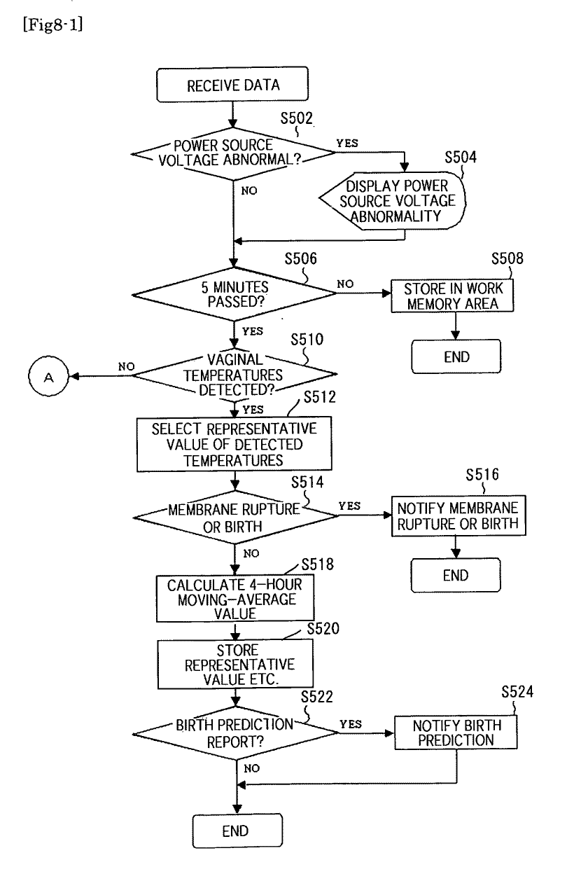

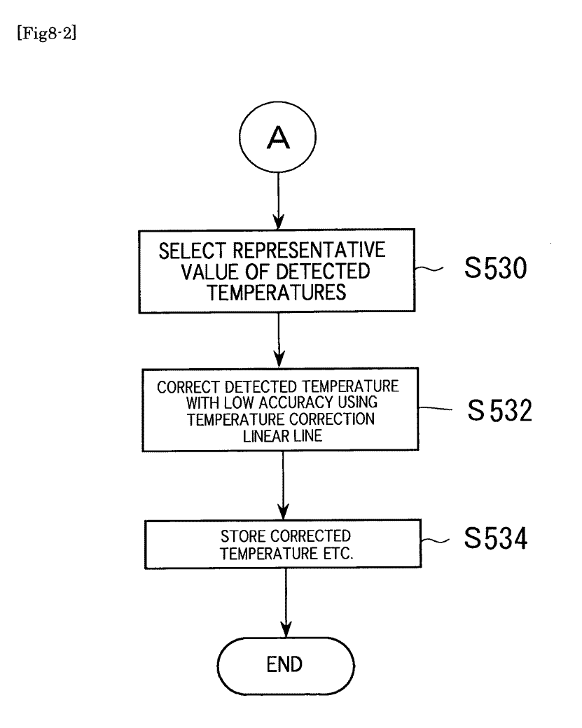

The processing shown in FIG. 8-1 in the case of temperature data sent from the temperature sensor/transmission modules 100 which measure another temperature instead of the temperature sensor/transmission modules 100 which measure vaginal temperature (NO in S510) is given in FIG. 8-2.

First, set the most frequent temperature data of multiple pieces of temperature data as a representative value of detected temperature with low accuracy (S530). Then correct the selected representative value of detected temperature with low accuracy using a temperature correction equation (S532). This shows that approximately 25 different classifications are possible if a characteristic of the detected temperature with low accuracy detected by each temperature sensor of the temperature sensor/transmission modules 100 is premeasured. Then store a value (fuse value) of 1 to 25 according to that characteristic in the fuse ROM 116 of the respective temperature sensor/transmission modules 100. A further accurate temperature may be obtained by incorporating this value within the ID in the transmission format, sending it, and correcting it using a correction equation as shown in FIG. 10 according to that value (fuse value).

As mentioned above, the reason that accurate birth prediction is possible with the present invention is because accurate measurement of cow vaginal temperature (0.1° C. resolution in the above working example) and detection that difference in 4-hour moving average is at least 0.3° C. lower is possible. Moreover, measurement of temperatures such as ambient temperature and water temperature is possible with the same structure.

### <Other Working Examples>

The temperature sensor/transmission module 100 used in the above working example is entirely covered by an elastic material, and has a Y shape with arms (two) on both sides. Alternatively, three or more arms to prevent the temperature sensor/transmission module 100 inserted in the vagina from escaping, as mentioned above, may be used. Moreover, a structure in which an escape preventing arm portion is separated from the main body including the temperature sensor/transmission module 100 is also possible.

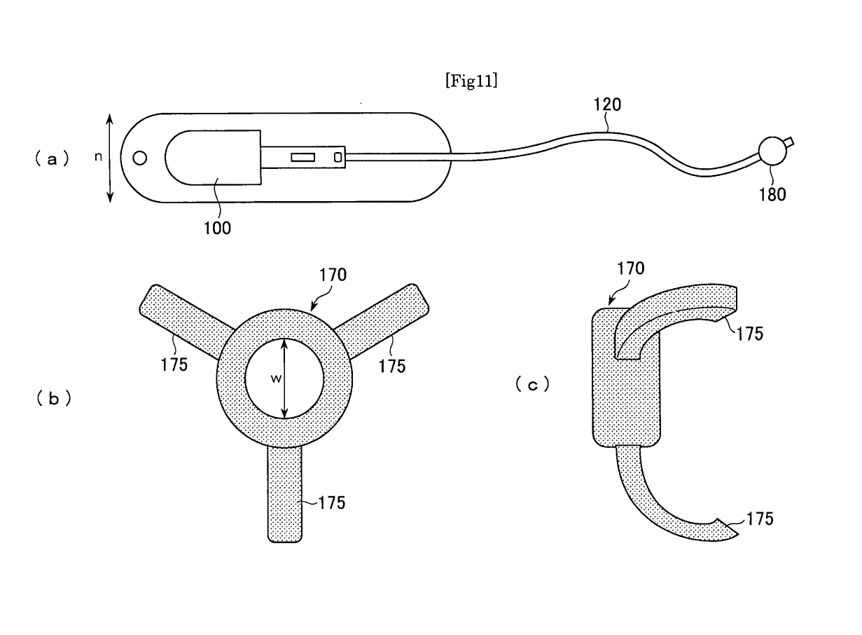

FIG. 11 shows an external appearance of a temperature sensor/transmission module 100 having a structure in which an escape preventing arm portion (stopper ring 170) is separated from the main body.

FIG. 11(a) is a diagram showing an external appearance of the main body including the temperature sensor/transmission module 100.

As shown in FIG. 11(a), the temperature sensor/transmission module 100 is entirely covered by an elastic material. Polypropylene resin, for example, is used as the elastic material. Moreover, although it is not expressed in FIG. 11(a), the cross section thereof is circular (column-shaped on the whole).

The antenna 120 extending in one direction from the main body has its conductive wire covered by an antenna covering tube. Moreover, a resin ball 180 for escape prevention is attached near the end of the far side from the antenna 120 main body.

The purpose of providing the resin ball 180 is to wrap the antenna 120 around the tail of the cow using a string or the like after the temperature sensor/transmission module 100 has been inserted into the cow's vagina. As a result, if the temperature sensor/transmission module 100 falls out of the cow's vagina for some reason, the resin ball 180 is caught where wrapped around, thereby preventing loss thereof by dropping out.

FIG. 11(b) is a top view of the stopper ring 170, and FIG. 11(c) is a side view of the stopper ring 170.

As shown in FIG. 11(b), the stopper ring 170 ring-shaped at the center and has three arms 175 (corresponding to the arms 160 shown in FIG. 2-1(a)). Moreover, as shown in FIG. 11(c), each of the arms 175 curves in the same direction.

The stopper ring 170 is made of an elastic material. Silicone resin, for example, is used as the elastic material.

The stopper ring 170 may be attached to the main body by inserting the main body into the stopper ring 170 (or fitting the stopper ring 170 to the main body).

Moreover, it is made such that the diameter of the cross-sectional circle of the main body (n in FIG. 11 (a)) and diameter of the inner side circle of the ring portion of the stopper ring 170 (w in FIG. 11(b)) have a relationship of n≧w. As a result, it may be securely fixed to the main body since it is made of an elastic material.

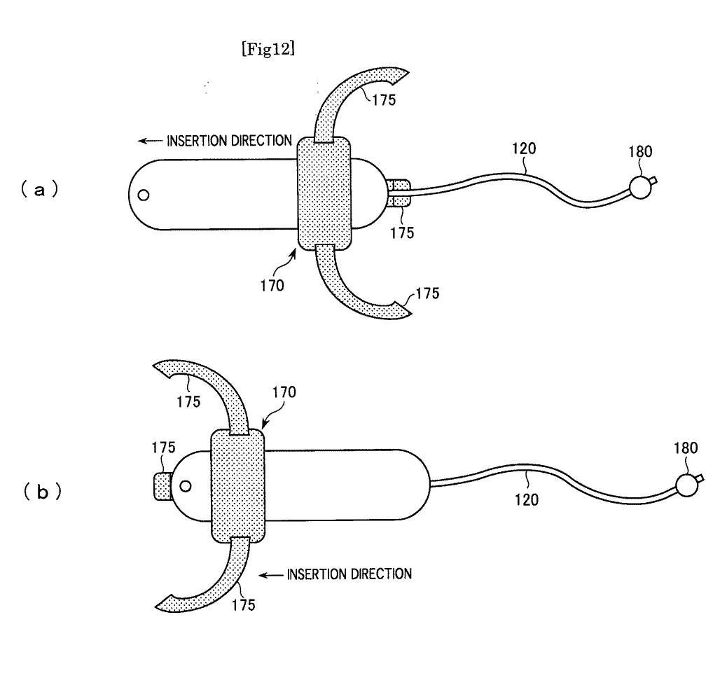

FIG. 12 shows an example of the stopper ring 170 fixed to the main body of the temperature sensor/transmission module 100.

FIG. 12(a) shows the state where the stopper ring 170 is fixed on the side close to the antenna of the main body. In this fixing example, the stopper ring 170 curves in the extending direction of the antenna of the main body.

When the temperature sensor/transmission module 100 is inserted in the cow vagina in the direction of the arrow shown in FIG. 12(a) in the state of this fixing example, the stopper ring 170 pressing the inner wall of the vagina makes it difficult for the temperature sensor/transmission module 100 to escape from the vagina.

As a result, in the case of use for prediction of birth, for example, even when birth is approaching and the vagina expands, the temperature sensor/transmission module 100 may be prevented from dropping out until membrane rupture or expulsion of a fetus.

Meanwhile, FIG. 12(b) shows the state where the stopper ring 170 is fixed on the side further from the antenna of the main body. In this fixing example, the stopper ring 170 curves in the opposite direction to the extending direction of the antenna of the main body.

When the temperature sensor/transmission module 100 is inserted in the cow vagina in the direction of the arrow shown in FIG. 12(b) in the state of this fixing example, extraction of the temperature sensor/transmission module 100 in the opposite direction to the insertion direction is easier.

As a result, in the case of use for follow-up observation of a disease, for example, the temperature sensor/transmission module 100 may be easily extracted from a vagina when it is temporarily inserted therein.

As described above, the temperature sensor/transmission module 100 of this working example is structured with the main body and the stopper ring 170 separated from each other. Therefore, it is possible to select the size of the stopper ring 170 for the main body in consideration of the purpose of vaginal temperature measurement (birth prediction, detection of coming into heat, follow-up observation, etc.) and build of the cow and related points, and freely adjust direction and position for fixation.

1-8. (canceled)

9. A birth prediction reporting system, comprising:
a temperature sensor/transmission module, which is inserted in the vagina of a cow, measures vaginal temperature at every predetermined time, and transmits wirelessly the measured vaginal temperature along with its own ID;
a reception module, which receives transmission data from the temperature sensor/transmission module and sends the received data through a communication line; and
a monitor center, which receives data from the reception module via the communication line, predicts birth based on the received vaginal temperature, and reports it, wherein
the temperature sensor/transmission module measures with high accuracy within a range from 34.1° C. to 44.0° C. near the average body temperature of cows, and
the monitor center measures a 4-hour moving-average of vaginal temperatures for each cow based on the ID, compares the moving-average with both of the 4-hour moving-average value obtained 24 hours before and 4-hour moving-average value obtained 48 hours before, carries out birth prediction when vaginal temperatures of both have decreased at least 0.3° C., and reports birth prediction results via the communication line.

10. The birth prediction reporting system of claim 1, wherein when the received temperature is detected to be outside of a predetermined vaginal temperature range, the monitor center determines the temperature sensor/transmission module being pushed out of the vagina due to membrane rupture or birth, and reports membrane rupture or birth via the communication line.

11. The birth prediction reporting system of either claim 1 or 2, wherein the temperature sensor/transmission module also measures a wide range of temperatures and transmits them wirelessly along with an identifier indicating a characteristic of temperature measurement, and
the monitor center corrects the sent temperature based on the identifier indicating the characteristic of temperature measurement.
